(19) United States
(12) Patent Application Publication
(10) Pub. No.: US 2021/0353353 A1
(43) Pub. Date: Nov. 18, 2021

(54) RF SHAVER CONNECTOR

(71) Applicant: GYRUS MEDICAL LIMITED, Cardiff (GB)

(72) Inventors: Nathan Philip BLAKE, Cardiff (GB); Rhydian HODDINOTT, Cardiff (GB)

(73) Assignee: GYRUS MEDICAL LIMITED, Cardiff (GB)

(21) Appl. No.: 17/206,368

(22) Filed: Mar. 19, 2021

(30) Foreign Application Priority Data
May 12, 2020 (GB) 2006970.4

(51) Int. Cl. A61B 18/14

(52) U.S. Cl. CPC A61B 18/14; A61B 2018/00178

(57) ABSTRACT

The present disclosure relates to an electrosurgical instrument, configured to be releasably connected to a hand-piece. The electrosurgical instrument comprises an elongate shaft, an end effector at a distal end of the elongate shaft, and an electrical connector interface. The electrical connector interface has at least one female electrical socket configured to receive in use at least one male electrical pin of the hand-piece. Prior to first use, the at least one female electrical socket is sealed from an exterior of the electrosurgical instrument by a pierceable sealing layer. The sealing layer is configured to be pierced by the at least one male electrical pins on first use. A further aspect of the present disclosure relates to an electrosurgical system comprising the electrosurgical instrument, the hand-piece and an electrosurgical generator.

### TECHNICAL FIELD

Embodiments of the present invention described herein relate to an electrosurgical device, and in particular to an electrosurgical device wherein a disposable instrument is connectable to a hand-piece via a connector, the connector facilitating an electrical and mechanical connection therebetween.

### BACKGROUND OF THE INVENTION

Electrosurgical instruments provide advantages over traditional surgical instruments in that they can be used for coagulation and tissue sealing purposes. One such prior art arrangement is known from U.S. Pat. No. 5,904,681 which describes a surgical instrument including a mechanical cutting portion, such as a rotary blade or burr, and a radio frequency (RF) cutting and/or cauterizing portion comprising an electronic surgical device which operates in bipolar mode.

Another prior art arrangement is known from U.S. Pat. No. 9,017,851 which describes an apparatus for powering a medical device, including a protective layer covering a battery pack and a connection feature. The connection feature provides a fluid tight seal, for example by having an electrode which may pierce the protective layer to establish electrical communication with the medical device from within the protective layer, thereby allowing a non-sterile battery pack to deliver power to a sterile medical device.

Another prior art arrangement is known from EP0746251 B1 which describes an integrated catheter assembly including a catheter having an irrigation fluid lumen, a distal tip portion having electrodes, and a needle extending through the catheter lumen and a lumen in the distal tip portion. The catheter is carried by a needle hub structure which provides a sealing entrance for a needle that can be displaced between extended and retracted positions while being electrically isolated.

Another prior art arrangement is known from US2009/221955A1 which describes an ablative apparatus having a distal end at which an ablation probe driven by a transducer may be vibrated to ablate tissues.

### SUMMARY OF THE INVENTION

Embodiments of the present invention provide an improved surgical instrument having an elongate shaft comprising an electrical connector interface and an end effector. The end effector may comprise a known end effector capable of different operations, including mechanical cutting of tissue and electrosurgical ablation, sealing and/or coagulation of tissue. The electrosurgical instrument can be connected to a hand-piece for provision of fluid, mechanical power and/or radiofrequency (RF) electrical signals. The electrical connector interface may be comprised in a hub of the instrument, the hub being configured to permit mechanical and electrical connection with the hand-piece. The electrical connector interface comprises at one least socket. The hand-piece comprises at least one electrical pin. The at least one socket may be defined by an electrical contact surface or may comprise an electrical contact arranged therein which can receive at least one electrical pin of the hand-piece. Each of the at least one sockets comprises a sealing layer configured to provide separation of the socket from the exterior of the electrical connector interface to thereby seal the electrical contact within the hub. The sealing layers can be pierced by the electrical pins when the hand-piece is brought into close contact with the electrical connector interface. A proximal end of the electrical pins comprises an electrically insulating portion. In this way, the electrical pins may be configured such that when fully inserted into the sockets, the entirety of the electrically conductive portion of an electrical pin is sealed within the hub by the sealing layer. This provides an electrical connection between the electrical pins and the electrical contacts while ensuring that the socket is protected from ingress of fluid or other matter, to thereby permit a clean and dry electrical connection between the electrosurgical instrument and the hand-piece, and isolate the electrical pins from one another to prevent a short circuit therebetween.

Advantageously, this arrangement permits an electrical connection between the electrosurgical instrument and the hand-piece such that the hand-piece provides the infrastructure required to provide electrical power to the electrosurgical instrument. This is an improvement over prior electrosurgical systems in which the hand-piece does not contain the infrastructure to provide electrical power to the electrosurgical instrument, thereby requiring the addition of an external cable which reduces the usability of the system. Such an external cable, in addition to increasing the number of components of the system, typically requires appropriate sealing with the electrosurgical instrument and/or hand-piece to prevent electrical short circuits, whereas the present arrangement integrates such requirements into the electrosurgical instrument itself.

In view of the above, from one aspect the present invention provides an electrosurgical instrument, configured to be releasably connected to a hand-piece, the electrosurgical instrument comprising:
an elongate shaft,
an end effector at a distal end of the elongate shaft, and
an electrical connector interface having at least one female electrical socket configured to receive in use at least one male electrical pin of the hand-piece,
wherein prior to first use the at least one female electrical socket is sealed from an exterior of the electrosurgical instrument by a pierceable sealing layer, the arrangement being such that the sealing layer is configured to be pierced by the at least one male electrical pins on first use.

Such an arrangement improves upon those known in the art by providing an electrosurgical instrument that may be powered using an integrated electrical connector interface, removing the need for an external cable. In this way, the device is more compact and can be operated more easily by a user. This advantage is realised without compromising the ingress protection requirements of such a device. In particular, the female electrical socket being sealed by a pierceable sealing layer prevents the ingress of moisture and other substances into the female electrical socket. This arrangement reduces the likelihood of a short circuit between the electrical pins of the hand-piece.

Further features and advantages of embodiments of the invention will be further apparent from the appended claims.

### BRIEF DESCRIPTION OF THE DRAWINGS

Further features and advantages of the present invention will become apparent from the following description of embodiments thereof, presented by way of example only, and by reference to the drawings, wherein:

FIG. 1 is a schematic diagram of an electrosurgical system including an electrosurgical instrument according to an embodiment of the present invention.

FIG. 2 is a perspective view of an electrosurgical system according to an embodiment of the present invention.

FIG. 3 is a further perspective view of a hand-piece of an electrosurgical system according to an embodiment of the present invention.

FIG. 4 is a side view of the electrosurgical instrument according to an embodiment of the present invention.

FIG. 5 is a perspective view of the electrosurgical instrument according to an embodiment of the present invention.

FIG. 6 is a side cross-sectional view of the electrosurgical system according to an embodiment of the present invention.

FIG. 7 is a further side cross-sectional view of the electrosurgical system according to an embodiment of the present invention.

FIG. 8 is a yet further side cross-sectional view of the electrosurgical system according to an embodiment of the present invention.

### DETAILED DESCRIPTION

An electrosurgical instrument is described herein in the context of an electrosurgical system for performing procedures such as ablation, sealing, resection and coagulation of tissues. The apparatus includes an electrosurgical generator capable of providing a mechanical and/or RF output. Such outputs may be controlled by various user inputs such as push buttons and switches, with the levels of various settings and other information being indicated on a display. The generator is connected to an electrosurgical device via a connection cord capable of transmitting power and RF signals thereto. Additionally, an irrigation and suction source is provided, capable of drawing fluid from, or providing fluid to, the electrosurgical device through tubes. The connection cord and tubes are connected to a hand-piece of the electrosurgical device. The hand-piece provides a means with which a practitioner may manipulate the electrosurgical device, and it may have further user input devices thereon for controlling the mechanical and/or RF output of the generator.

The hand-piece comprises a connecting portion to permit the electrosurgical instrument to releasably connect therewith. The connecting portion includes male electrical pins, while the electrosurgical instrument comprises a hub having female electrical contacts therein. The female electrical contacts can be configured to surround at least a portion of the male electrical pins when they are brought into contact with one another. The female electrical contacts are comprised within sockets of the hub at an electrical connector interface of the electrosurgical instrument. At least one socket is covered by a deformable sealing layer to seal the interior of the socket from the exterior. The electrical connector interface is configured such that when the male electrical pins of the hand-piece are brought towards the sockets, the male electrical pins pierce the sealing layer, creating a void therein, to allow the male electrical pins to access the female electrical contacts in the interior of the sockets, to thereby permit electrical connection between the male electrical pins and the female electrical contacts. In particular, the sealing layer provides a means to seal the interior of the sockets to protect it from ingress originating from the exterior of the hub, such as fluid ingress, while permitting entry of the male electrical pins via the void. The sealing layer is arranged such that upon removal of the male electrical pins from the sockets, the void in the sealing layers closes up such that the sealing layers continue to provide ingress protection to the interior of the sockets. Furthermore, upon re-insertion of a male electrical pin into a socket, the pierced sealing layer may act as a wiper to permit cleaning and drying of an electrical pin as it enters the socket.

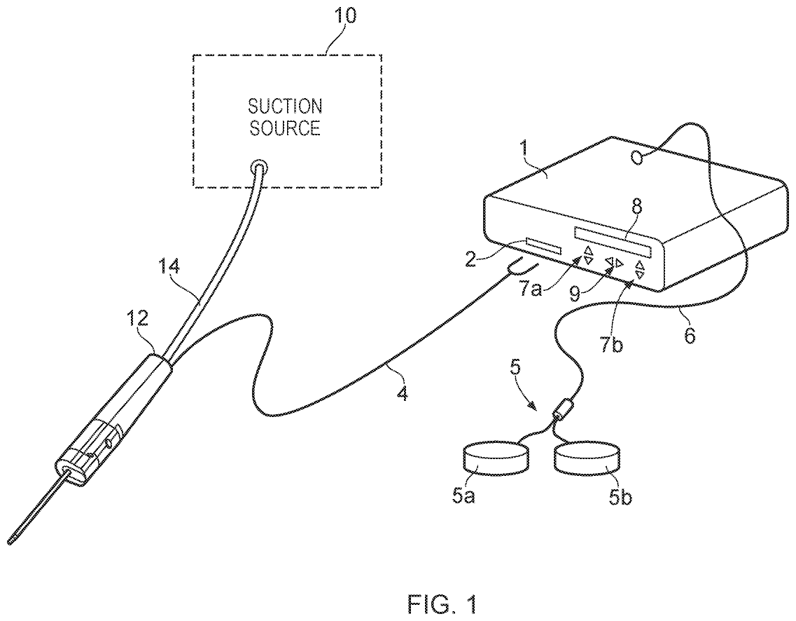

Referring to the drawings, FIG. 1 shows electrosurgical apparatus including an electrosurgical generator 1 having an output socket 2 providing a radio frequency (RF) output, via a connection cord 4, for an electrosurgical device 12. The device 12 has irrigation and suction tubes 14 which are connected to an irrigation fluid and suction source 10. Activation of the generator 1 may be performed from the device 12 via a hand-switch (not shown) on the device 12, or by means of a footswitch unit 5 connected separately to the rear of the generator 1 by a footswitch connection cord 6. In the illustrated embodiment, the footswitch unit 5 has two footswitches 5a and 5b for selecting a coagulation mode or a cutting or vaporisation (ablation) mode of the generator 1 respectively, although in some embodiments of the electrosurgical device 12 described herein it is envisaged that only one or other of the coagulation or ablation modes would be used, with cutting being provided mechanically by way of a rotating tube having a sharpened cut-out portion. The generator front panel has push buttons 7a, 7b for respectively setting ablation (cutting) or coagulation power levels, which are indicated in a display 8. Push buttons 9 are provided as an alternative means for selection between the ablation (cutting) and coagulation modes.

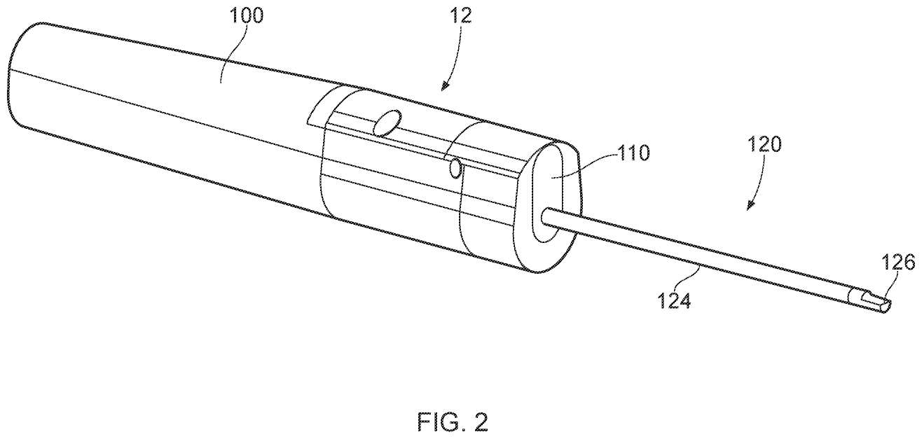

FIG. 2 shows the electrosurgical device 12 that forms the basis of an embodiment of the present invention. The electrosurgical device 12 comprises a hand-piece 100 and an electrosurgical instrument 120 fully inserted into the hand-piece 100. The hand-piece 100 may also comprise activation buttons (not shown) to permit an operator to activate the mechanical cutting or electrosurgical operations of the device. The instrument 120 comprises an elongate shaft 124 having an end effector assembly 126 at a distal end and a hub 110 at a proximal end. In the illustrated example, the hub 110 is fixed around the shaft 124 and is itself releasably connected to the hand-piece 100. In this way, the instrument 120 is releasably connectable to the hand-piece 100. While the hand-piece 100 is described above as being supplied power via an electrosurgical generator 1, it will be appreciated that mechanical drive and/or RF signals may be provided from the hand-piece itself as part of an integral component thereof, without requiring an electrosurgical generator for operation.

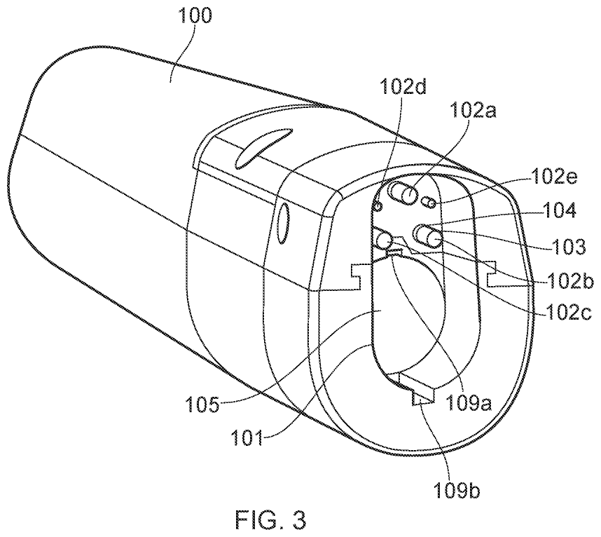

FIG. 3 shows the hand-piece 100, without the electrosurgical instrument 120. The hand-piece 100 comprises a connecting portion 101 at a distal facing end. In the illustrated example, the connecting portion 101 is recessed within the hand-piece 100. The connecting portion 101 comprises at least one male electrical pin 102 projecting from a distal facing surface of the connecting portion 101. In the illustrated example, the connecting portion 101 comprises five male electrical pins 102a, 102b, 102c, 102d, 102e arranged in a pentagonal fashion. Three of the male electrical pins 102a-102c have a greater diameter and project further from the connecting portion 101 compared to the remaining two male electrical pins 102d, 102e. Referring to one particular male electrical pin 102a, there is provided an electrically conductive portion 103 at a distal end of the male electrical pin 102a and an electrically insulative portion 104 at a proximal end of the male electrical pin 102a. Equivalent electrically conductive and electrically insulative portions may be provided on each of the remaining male electrical pins 102b-102e. The hand-piece 100 further comprises a bore 105, having a top groove 109a and a bottom groove 109b.

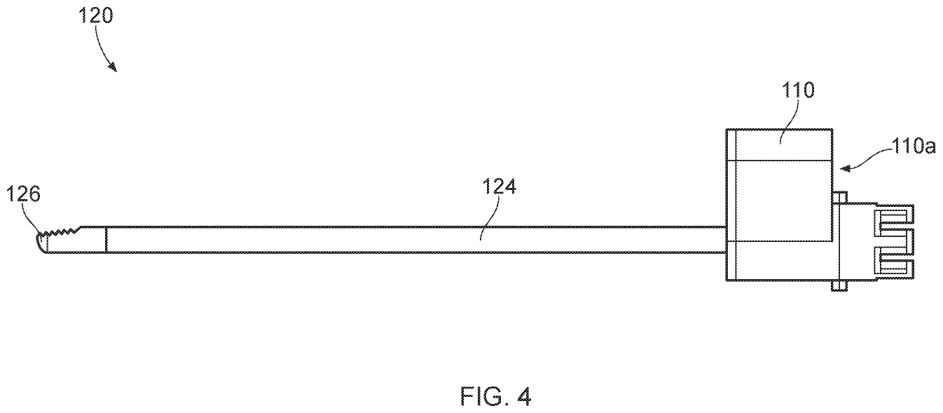

FIG. 4 shows the electrosurgical instrument 120 without the hand-piece 100. The electrosurgical instrument 120 comprises an elongate shaft 124. An electrical connector interface 110a is provided at a proximal end of the elongate shaft 124. The electrical connector interface 110a may be comprised in a hub 110. An end effector 126 is provided at a distal end of the elongate shaft 124. The end effector 126 may comprise at least one electrode (not shown) for ablation and/or coagulation procedures, and may instead or additionally comprise at least one cutting blade (not shown) for mechanical cutting procedures. At least one cutting surface of a cutting blade may be arranged concentrically within the end effector 126 to permit its rotation about an axis of the end effector 126.

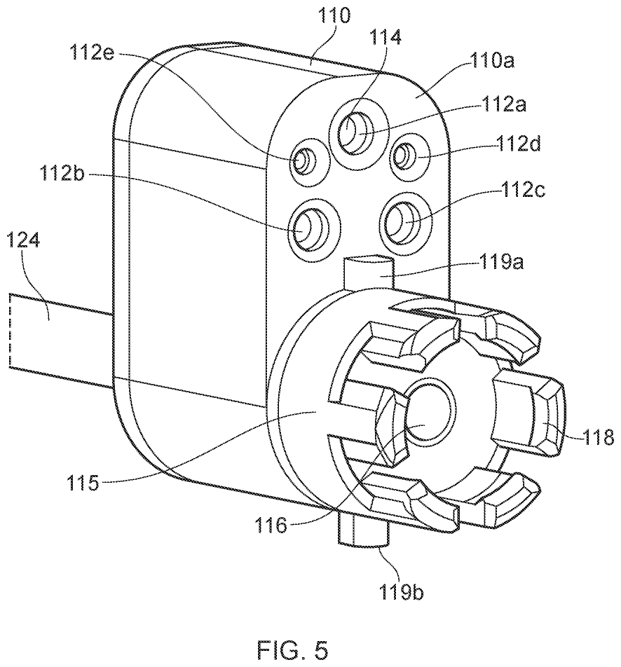

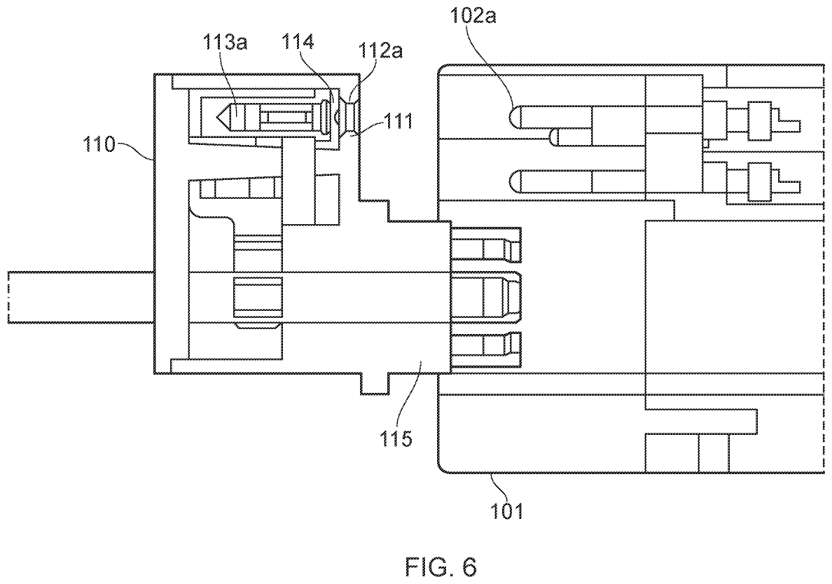

FIG. 5 shows the proximal end of the electrosurgical instrument 120 and in particular shows further details of the electrical connector interface 110a of the hub 110. The shaft 124 can be received in a circular hole 116 of the hub 110. The electrical connector interface 110a comprises at least one socket 112, having an entrance facing the proximal direction of the electrosurgical instrument 120. In the illustrated example, the electrical connector interface 110a comprises five such sockets 112a, 112b, 112c, 112d, 112e arranged in a pentagonal fashion corresponding to that of the male electrical pins 102a-102e. The entrance to three of the sockets 112a-112c has a greater diameter than the entrance of the remaining two sockets 112d, 112e. In the illustrated example, the hub 110 further comprises a circular plug 115 extending from the hub 110 in the proximal direction of the electrosurgical instrument 120. The plug 115 is arranged on the hub 110 in a position that corresponds to the bore 105 of the hand-piece 100, to be received therein. The plug 115 may comprise alignment means 118, which in this illustration is shown as a set of teeth axially extending from the plug 115 in the proximal direction of the instrument 120. The plug 115 may further comprise a top tongue 119a and a bottom tongue 119b arranged radially around the plug 115 in a position that corresponds to that of the top and bottom grooves 109a, 109b of the hand-piece 100. There is provided a sealing layer 114 at the entrance of at least one of the sockets 112. The sealing layer 114 may be a soft polymer septa gasket and may be an elastomer. In the illustrated example, each of the sockets 112a-112e comprises a sealing layer 114 at its entrance. Further details of the sealing layer 114 will be described in relation to FIG. 6 below.

FIG. 6 shows a cross-sectional view of the hub 110 of the instrument 120 and the connecting portion 101 of the hand-piece 100. The hub 110 and the hand-piece 100 are shown to be arranged such that the plug 115 is partially inserted into the connecting portion 101. Referring to one particular socket 112a, there is provided a corresponding electrical contact 113a. In the illustrated example, the socket 112a is an elongate chamber in the hub 110. The elongate chamber has a reduced-diameter opening defined by a rim 111. The electrical contact 113a may be a female electrical contact arranged within the socket 112a. The sealing layer 114 may be provided at the entrance to the socket 112a between the electrical contact 113a and the opening of the socket 112a. In particular, the sealing layer may be provided between the electrical contact 113a and the rim 111. The sealing layer 114 may extend around at least a portion of an interior longitudinal surface of the socket 112a. The foregoing description may also apply to the remaining sockets 112b-112e in an equivalent manner.

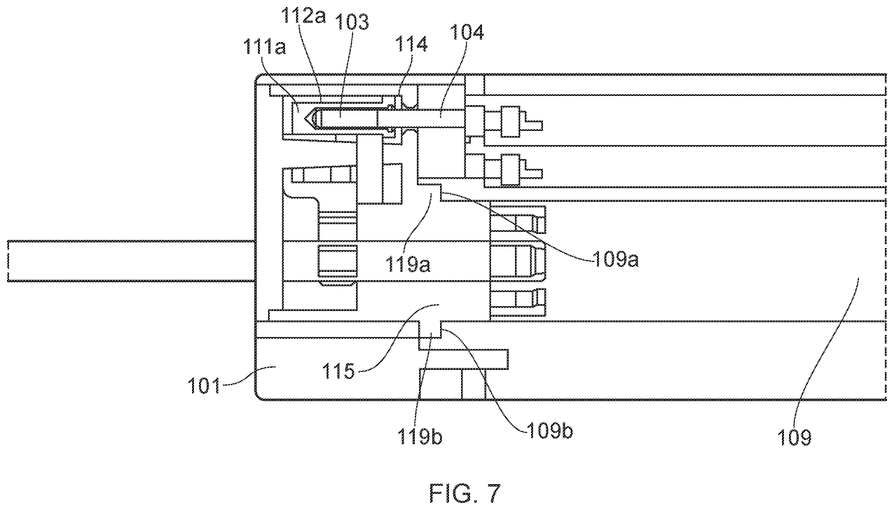

FIG. 7 shows a further cross-sectional view of the hub 110 and the connecting portion 101 shown such that the hub 110 is fully inserted into the connection portion 101. In this arrangement, the plug 115 can fit inside the bore 105 while the tongues 109a, 109b may slide along the grooves 119a, 119b to secure the axial and radial position of the electrical connector interface 110a with respect to the hand-piece 100. Upon bringing the electrical connector interface 110a towards the connecting portion 101 of a particular electrosurgical instrument 120, the electrical pin 102a can be inserted into the socket 112a to be connected with the electrical contact 113a. Upon insertion of the electrical pin 102a into the socket 112a for the first time, the sealing layer 114 may be pierced by the electrical pin 102a to thereby allow the electrical pin 102a to access to the socket 112a. As the electrical pin 102a is fully inserted into the socket 112a, the sealing layer 114 can act as a radial seal around the electrical pin 102a. Furthermore, the sealing layer 114 can act as a wiper seal to prevent ingress from non-sterile substances into the socket 112a.

The electrical pin 102a may be arranged such that when the electrical connector interface 110a is fully inserted into the hand-piece 100, the entirety of the electrically conductive portion 103 is sealed within the socket 112a by the sealing layer 114. The electrical pin 102a may be electrically connected to the hand-piece 100 through a wire (not shown) provided inside the electrically insulative portion 104 of the electrical pin 102a. In this way, the electrical pin 102a may be brought into contact with the electrical contact 113a while preventing potentially non-sterile substances from entering the socket 112a, thereby permitting a dry and clean connection between the electrical pin 102a and the electrical contact 113a. The foregoing description can apply equivalently to the sockets 112b-112e having female electrical contacts 113b-113e. Therefore, another advantage of this arrangement can be seen in that the electrically conductive portion 103 of a particular electrical pin 102 is sealed inside its respective socket from the other electrical pins 102, thereby reducing the likelihood of short circuits between multiple electrical pins 102.

Upon removal of the electrical connector interface 110a from the hand-piece 100, the electrical pins 102a-102e are removed from their corresponding sockets 112a-112e and brought out of contact with the electrical contacts 113a-113e. Once an electrical pin 102 is fully removed from a socket 112, the elastic nature of the sealing layer 114 may cause it to close up, thereby sealing the socket 112 from the exterior of the instrument to prevent ingress of moisture or other substances. Upon reinsertion of an electrical pin 102 into a socket 112, the sealing layer 114 may act as a wiper seal to remove substances from the electrical pin 102 upon its entrance into the socket 112. In this way, even for subsequent reinsertions of the electrical connector interface 110a into the hand-piece 100, the sealing layer 114 seals the electrically conductive portion 103 of the at least one male electrical pins 102 to provide ingress protection and to prevent short circuiting.

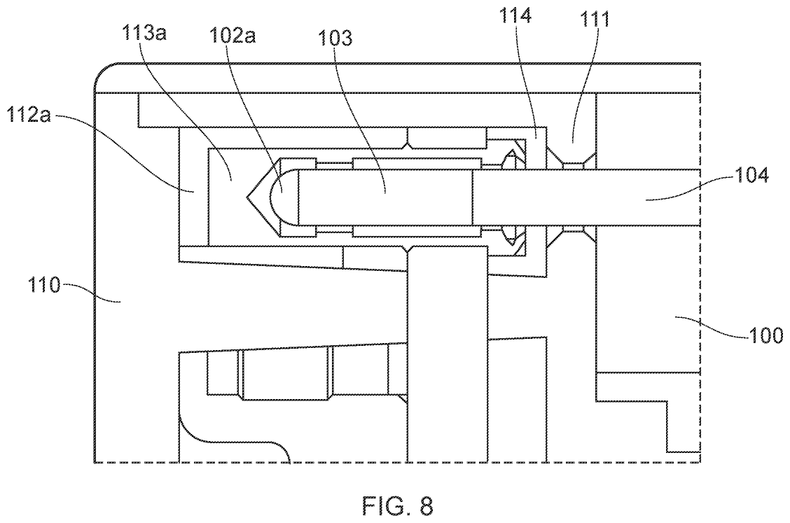

FIG. 8 shows further details of a socket 112a of the electrical connector interface 110a of the hub 110. In particular, FIG. 8 illustrates the surfaces on which the sealing layer 114 seals. In the illustrated example, the sealing layer 114 can act as a radial seal to provide sealing around an outer circumferential surface of the electrical pin 112a, particularly around the electrically insulative portion 104 of the electrical pin 112a. Furthermore, the sealing layer 114 can provide sealing around a surface of the rim 111 to prevent fluid or other substances from circumventing the sealing layer 114. The sealing layer 114 may be provided as a separate sealing layer 114 for each of the at least one sockets 112, with each sealing layer comprising a soft polymer septa gasket. In an alternative arrangement, the sealing layer 114 may be provided as a single layer configured to seal all of the sockets 112 simultaneously, such as by providing a mould of soft polymer over the hub 110. While the sealing layer 114 is shown to be located at in interior position of the socket 112a, it will be appreciated that the sealing layer 114 may be located on an exterior surface of the electrical connector interface 110a, provided as a single sealing layer or a plurality of separate sealing layers. Furthermore, an alternative arrangement may comprise more than one sealing layer per socket.

Various modifications, whether by way of addition, deletion and/or substitution, may be made to all of the above described embodiments to provide further embodiments, any and/or all of which are intended to be encompassed by the appended claims.

1. An electrosurgical instrument, configured to be releasably connected to a hand-piece, the electrosurgical instrument comprising:
an elongate shaft,
an end effector at a distal end of the elongate shaft, and
an electrical connector interface having at least one female electrical socket configured to receive in use at least one male electrical pin of the hand-piece,
wherein prior to first use the at least one female electrical socket is sealed from an exterior of the electrosurgical instrument by a pierceable sealing layer, the arrangement being such that the sealing layer is configured to be pierced by the at least one male electrical pins on first use.

2. The electrosurgical instrument according to claim 1, configured such that, in use, the sealing layer provides a fluid tight seal around the at least one male electrical pin.

3. The electrosurgical instrument according to claim 1, configured such that, after removal of the at least one male electrical pin from the at least one female electrical socket, the sealing layer closes to seal the at least one female electrical socket from the exterior of the electrosurgical instrument.

4. The electrosurgical instrument according to claim 1, wherein the sealing layer is a septa gasket.

5. The electrosurgical instrument according to claim 1, wherein the sealing layer comprises a polymeric material, preferably an elastomeric material.

6. The electrosurgical instrument according to claim 1, wherein the at least one socket comprises at least one electrical contact, the sealing layer being provided between the at least one electrical contact and an opening of the at least one female electrical socket.

7. The electrosurgical instrument according to claim 1, wherein the end effector comprises at least one cutting surface and/or at least one electrode in order to cut, ablate and/or coagulate tissue

8. An electrosurgical system comprising a hand-piece and an electrosurgical instrument, the electrosurgical instrument comprising:
an elongate shaft,
an end effector at a distal end of the elongate shaft, and
an electrical connector interface having at least one female electrical socket configured to receive in use at least one male electrical pin of the hand-piece,
wherein prior to first use the at least one female electrical socket is sealed from an exterior of the electrosurgical instrument by a pierceable sealing layer, the arrangement being such that the sealing layer is configured to be pierced by the at least one male electrical pins on first use;
the hand-piece comprising at least one male electrical pin configured to correspond with the at least one female electrical socket.

9. The electrosurgical system according to claim 8, wherein at least one male electrical pin comprises an electrically conductive portion configured such that, in use, the electrically conductive portion is sealed within the female electrical socket by the sealing layer.

10. The electrosurgical system according to claim 9, wherein at least one male electrical pin further comprises an electrically insulative portion configured such that, in use, the sealing layer seals around an outer circumferential surface of the electrically insulative portion.

11. The electrosurgical system according to claim 8, and further comprising a radiofrequency electrosurgical generator.

12. The electrosurgical system according to claim 11, wherein the radiofrequency electrosurgical generator is comprised within the hand-piece as an integral part thereof.

13. An electrosurgical instrument, configured to be connectable to an RF source of electrosurgical signals, the electrosurgical instrument comprising:
an electrosurgical end effector having electrical connections, and
an electrical connector interface having at least one female electrical socket configured to receive in use at least one male electrical pin of the RF source,
wherein prior to first use the at least one female electrical socket is sealed from an exterior of the electrosurgical instrument by a pierceable sealing layer, the arrangement being such that the sealing layer is configured to be irreversibly broken by the at least one male electrical pin on first connection of the electrosurgical instrument to the RF source.
